(19) United States
(12) Patent Application Publication
(10) Pub. No.: US 2021/0121511 A1
(43) Pub. Date: Apr. 29, 2021

(54) VIRUS-LIKE PARTICLE COMPOSITIONS AND METHODS OF USING SAME

(71) Applicants: Henry C. Lin, Albuquerque, NM (US); Derek M. Lin, Albuquerque, NM (US); Britt Koskella, Berkeley, CA (US); UNM RAINFOREST INNOVATIONS, Albuquerque, NM (US); UNIVERSITY OF CALIFORNIA, BERKELEY, Berkeley, CA (US)

(72) Inventors: Henry C. Lin, Albuquerque, NM (US); Derek M. Lin, Albuquerque, NM (US); Britt Koskella, Berkeley, CA (US)

(21) Appl. No.: 17/058,565

(22) PCT Filed: May 17, 2019
(86) PCT No.: PCT/US2019/032784

§ 371 Date: Nov. 24, 2020

Related U.S. Application Data
(60) Provisional application No. 62/677,425, filed on May 29, 2018.

(51) Int. Cl. A61K 35/76; A61K 31/702; A61K 31/733; A61K 35/74

(52) U.S. Cl. CPC A61K 35/76; A61K 35/74; A61K 31/733; A61K 31/702

(57) ABSTRACT

A pharmaceutical composition includes a preparation of virus-like particles (VLPs) obtained from the gastrointestinal tract of a subject and a pharmaceutically-acceptable carrier. The VLPs can include bacteriophages, eukaryotic viruses, or gene transfer agents. A VLP composition may be administered to a subject having, or at risk of having dysbiosis, in an amount effective to amelio-rate at least one symptom or clinical sign of dysbiosis. A VLP composition also may be administered to a subject in preparation for administering a source of bacteria, to improve the receptivity of subject's gut to source of bacteria.

### CROSS-REFERENCE TO RELATED APPLICATION

This application claims the benefit of U.S. Provisional Patent Application No. 62/677,425, filed May 29, 2018, which is incorporated herein by reference in its entirety.

### SUMMARY

This disclosure describes, in one aspect, a pharmaceutical composition that includes a preparation of virus-like particles (VLPs) obtained from the gastrointestinal tract of a subject and a pharmaceutically-acceptable carrier.

In some embodiments, the pharmaceutical composition can further include an adjuvant.

In some embodiments, the VLPs can include bacteriophages, eukaryotic viruses, fungal viruses, archaeal viruses, gene transfer agents, or any combination of two more thereof.

In another aspect, this disclosure describes a method of treating dysbiosis in a subject having, or at risk of having dysbiosis. Generally, the method includes administering to the subject a virus-like particle (VLP) preparation in an amount effective to ameliorate at least one symptom or clinical sign of dysbiosis.

In some embodiments, the method further includes administering to the subject a second pharmaceutical composition for treating dysbiosis. In some of these embodiments, the second pharmaceutical composition for treating dysbiosis comprises an antibiotic, a prebiotic, a probiotic, a synbiotic, a microbiota transplant, a pharmaceutical agent, a non-pharmaceutical pharmacological agent, a nutraceutical, a nutritional supplement, a biofilm modifier, a biofilm emulsifier, an autophagy regulator, a phage-encoded protein, a dietary treatment, phage therapy, immunoglobulin therapy, interferon-gamma therapy, growth factor therapy, CRISPR-Cas9, or a stem cell transplant.

In some embodiments, the dysbiosis may be localized to an epithelial tissue. In some of these embodiments, the epithelial tissue can include epidermis, a mucosal surface, at least a portion of the skin or scalp, at least a portion of the oral cavity including tooth or gum, at least a portion of the gastrointestinal tract, at least a portion of the nasal cavity, at least a portion of the respiratory tract, at least a portion of the genito-urinary tract, or a body cavity.

In some embodiments, the VLP preparation is incorporated into a medical device. In some of these embodiments, the medical device can include a pacemaker, a catheter, or a stent.

In some embodiments, the dysbiosis causes bacterial overgrowth.

In some embodiments, the VLP preparation is prepared from an environmental sample. In some of these embodiments, the environmental can include salt water, soil, fresh water, sewage, activated sludge, hospital effluent, wastewater, treated wastewater, swamp water, marsh water, brackish water, lake water, pond water, stream water, river water, or a deep-sea vent.

In some embodiments, the method further includes a sustained period of nutrient deprivation of epithelial bacterial population prior to administering the VLP preparation to the subject.

In another aspect, this disclosure describes a method of preparing or priming the gut environment of a healthy subject for administering a source of bacteria. Generally, the method includes administering to the subject a virus-like particle (VLP) preparation in an amount effective to improve the receptivity of subject's gut to source of bacteria.

In some embodiments, the source of bacteria can include a probiotic or a fecal microbiota transplant.

In some embodiments, the method further includes a sustained period of nutrient deprivation of the subject's gut bacterial population prior to administering the VLP preparation to the subject.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. A framework for the dysbiosis recovery system. This figure illustrates the role of the host-associated microbiome in mediating the transition between homeostasis (i.e., health) and dysbiosis (i.e., disease) within the host. While the disclosure describes specific conditions to illustrate the methods and compositions of the invention, this dysbiosis recovery system is suitable for use in any host and for any host-associated microbiome.

FIG. 2. HFD-treated animals who received a PBS treatment (HFD-PBS) had a 3.2-times higher Firmicutes-Bacteroidetes ratio in the ileal microbiome compared to SD-PBS Control animals (HFD-PBS=6.3±5.7; SD-PBS=2.0±3.6) (p<0.05), demonstrating dysbiotic microbiome composition. However, the Firmicutes-Bacteroidetes ratio in HFD-treated animals who received the VLP solution (HFD-VLP=1.2±0.9) was not significantly different from the Control animals (p=0.99). All results were represented as ratio of 16 s rRNA gene copies associated with Firmicutes to that of Bacteroidetes from that of the Control group (at 1.0). (*p<0.05).

FIG. 3. HFD-treated animals who received a PBS treatment (HFD-PBS) had a 3.2-fold increase in bacterial density compared to Control animals (p<0.05) demonstrating diet-induced bacterial overgrowth. However, the bacterial density in HFD-treated animals who received the VLP solution (HFD-VLP) was significantly less than the HFD-PBS group (p<0.05) and was not significantly different from the Control animals (p=0.8). All results were represented as fold change of 16 s copy number from that of the Control group (at 1.0). (*p<0.05).

FIG. 4. Weighted UniFrac distances of beta diversity (i.e., bacterial community composition) plotted on the principal coordinates analysis plot. HFD-treated animals who received a PBS treatment (HFD-PBS) had altered beta diversity of the bacterial community compared to SD-treated Control animals (SD-PBS) (p<0.01), as indicated by clustering on the principal coordinates analysis plot. The separate clustering demonstrates dysbiosis of the gut bacterial community in the HFD-PBS group. Treatment of SD animals with a VLP solution derived from an HFD donor transitioned the bacterial community of SD animals towards that of the HFD cluster.

FIG. 5. HFD-treated animals who received a PBS treatment (HFD-PBS) had a 0.3-times the expression of the Muc2 gene compared to control animals (p<0.05) demonstrating an impaired mucus layer. However, Muc2 gene expression in HFD-treated animals who received the VLP solution (HFD-VLP) was significantly higher than the HFD-PBS group (p<0.05) but was not significantly different from the Control animals (p=0.9). All results were represented as quantity of Muc2 mRNA transcripts relative to that of the Control group (at 1.0). (*p<0.05).

FIG. 6. Images of VLPs extracted from feces for transplantation into recipients. Identified using electron microscopy. Scale bars are indicated at bottom.

### DETAILED DESCRIPTION OF ILLUSTRATIVE EMBODIMENTS

Microbiomes are ubiquitous communities of microorganisms (i.e., bacteria, archaea, fungi, viruses, and other microscopic biological entities) that are associated with a host organism, environmental niche, or ecosystem. A growing body of evidence suggests that the composition of the microbiome can directly influence a host's susceptibility to disease. Disease-associated microbiome compositions, termed “dysbiosis,” promote inflammation, both within the tissue where the dysbiotic microbiome resides and systemically. During dysbiosis of the gut microbiome, there is an expansion of some members of the microbiota, specifically in the phylum Proteobacteria. Bacterial species in the Proteobacteria phylum degrade the protective mucus layer on the gastrointestinal epithelium which allows luminal contents (e.g., bacterial endotoxins) from the gastrointestinal tract access to proinflammatory receptors on the surface of epithelial cells. Inflammation of the epithelium changes the morphology of the tissue and mucus layer, impairing the function of the intestinal barrier, and leading to increased intestinal permeability, or “leaky gut.” Leaky gut allows for translocation of bacteria, their endotoxins, and other luminal contents into the host's circulation, thereby promoting systemic inflammatory response.

Within the human gastrointestinal tract, the gut microbiome has been found to be associated with numerous clinical disorders and disease states such as inflammatory bowel disease (IBD), colorectal cancer, and multiple sclerosis. During states of health, the relationship between microbiome and host is a mutualism, where both parties benefit from the other (i.e. homeostasis). During states of disease, the microbiome behaves pathogenically, causing harm to the host (i.e. dysbiosis). The nature of the relationship between gut microbiome and its human host is determined by environmental factors including, but not limited to, host diet, medication, psychological stress, exercise, and genetics. While described herein in the context of the mammalian gut microbiome, dysbiosis can occur in the microbiomes of either plant or animal in any tissue or organ.

Within gut-associated microbiomes, dysbiosis can be caused by stressors such as a “Western” diet (i.e., high fat, high sugar, low fiber) and is typically characterized by a reduction in bacterial diversity, richness, or evenness in the microbiome and an increased density of some bacterial members when compared to healthy individuals. Other forms of dysbiosis take place in the eukaryotic, archaeal, fungal, or viral communities of the microbiome. Herein, dysbiosis will be described in terms of the bacterial community but applies to all microbial communities—i.e., eukaryotic, archaeal, fungal, or viral. Dysbiosis in the gut can be identified using 16 s rRNA sequencing to assess the microbiome composition for: increased or decreased ratio of the bacterial phyla Firmicutes and Bacteroidetes, an expansion in the phylum Proteobacteria, or a dysregulation in host control of the bacterial community that results in increased density of some or all bacterial members (i.e. small intestinal bacterial overgrowth). Dysbiosis also can be identified in the host by looking for inflammatory markers and impaired epithelial function (e.g., reduced mucin expression). In contrast to classic bacterial infections involving a single bacterial pathogen, such as E. coli in urinary tract infections, dysbiosis may involve the entire diverse membership of the gut microbiome, which includes all cellular microorganisms including resident (i.e., non-pathogenic bacteria), eukaryotic microorganisms, archaea, and fungi. In natural environments, each of these cellular microorganisms are regulated by their respective viruses. Viruses infect all cellular microorganisms and have a role of regulating the populations of their hosts, thereby limiting population density and supporting diversity. All viruses within a mixed microbial community (i.e. microbiome) are collectively called virus-like particles (VLP). VLPs include bacteriophages (phages or bacterial viruses), archaeal viruses, eukaryotic viruses, fungal viruses, and gene transfer agents. All naturally-occurring microbial communities have associated VLP communities.

Current treatments for dysbiosis include medication, change of diet, and fecal microbiota transplantation. While none of these treatments work all of the time, all treatment options have the intention of modifying the resident microbiome and driving it towards a state of homeostasis.

In contrast, this disclosure describes compositions and methods for treating dysbiosis that involve the use of virus-like particles (VLPs). VLPs, as natural regulators of cellular microorganisms, have the advantage of being self-replicating and each VLP in the community of VLPs of the composition is highly specific to its respective host.

Dysbiosis can take place in any host-associated microbiome, can be caused by a range of environmental perturbations, and presents with a variety of characteristics and consequences for the host. As such, this disclosure describes a dysbiosis recovery system designed to restore homeostasis for any manifestation of dysbiosis. While described, for illustrative purposes, in the context of specific conditions relating to gut microbiome dysbiosis, this system and methods described herein are designed to be universally applicable to any manifestation of dysbiosis and readily modifiable for treating dysbiosis in contexts other than dysbiosis of the gut microbiome.

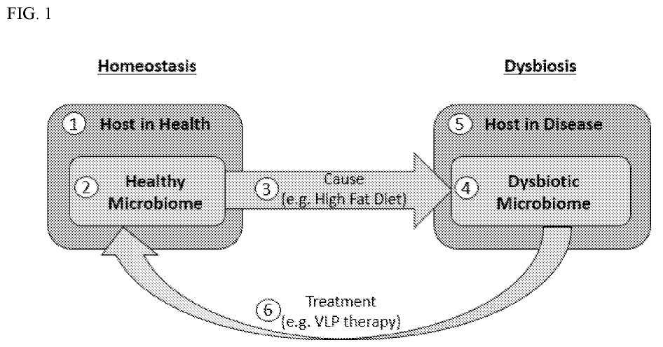

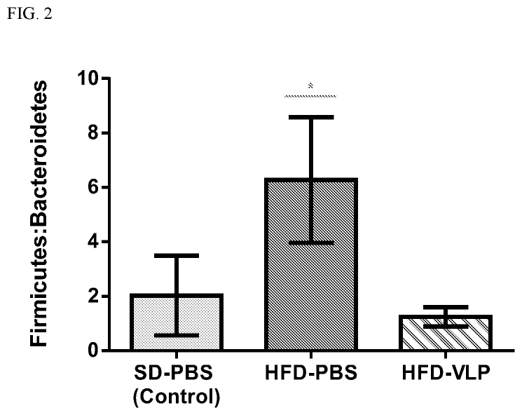

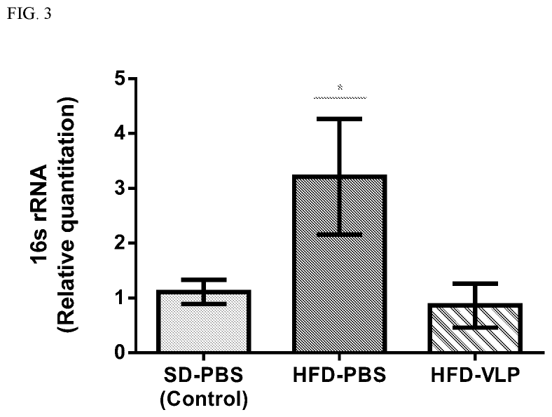

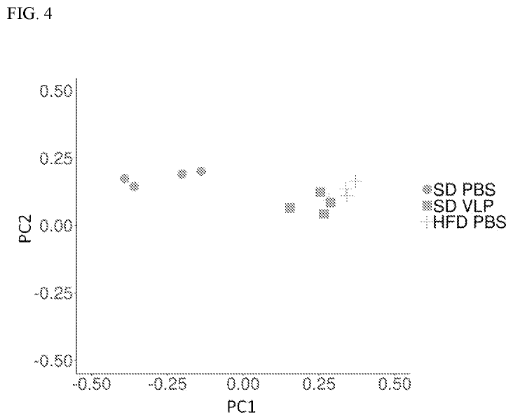

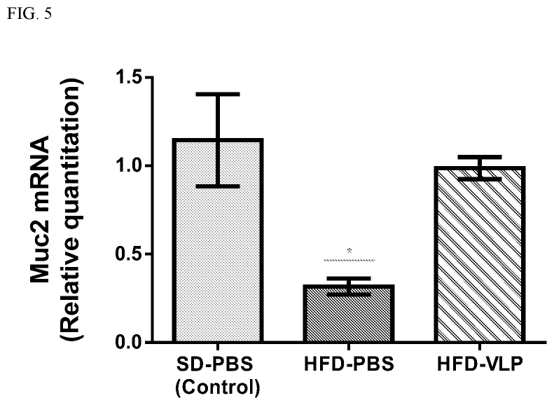

FIG. 1 provides a schematic illustration of various aspects of treating dysbiosis. The dysbiosis recovery system is exemplified herein using a mouse model to induce dysbiosis in the small intestinal microbiome using a high-fat diet (HFD). Dysbiosis in this setting was characterized by an increased Firmicutes-Bacteroidetes ratio (FIG. 2, HFD-PBS), an increased bacterial density (FIG. 3, HFD-PBS), and an altered beta diversity (FIG. 4, HFD-PBS). Signs of disease in the mouse host included a reduced expression of Muc2 (FIG. 5, HFD-PBS) and a weakening of defenses that allowed for increased bacterial colonization in the small intestine (FIG. 3, HFD-PBS).

Homeostasis was restored by reversing these characteristics of dysbiosis using a mixed population of virus-like particles (VLP) from feces. As used herein, VLPs are nucleic acid containing particles in the range of 3 nm to 450 nm in diameter that are dependent on either prokaryotic or eukaryotic host cells for reproduction. VLPs, collectively, can include phages, eukaryotic viruses, gene transfer agents (GTA), and/or any combination or subcombination thereof. Thus, the term “virus-like particle” refers to a viral member from a mixed microbial community. Accordingly, a VLP, as used herein, need not be engineered or otherwise modified to reduce infectivity, as may be the case in other fields (e.g., immunology).

VLPs shape the composition of microbial populations by regulating their respective cellular hosts (e.g., phages regulate bacteria, fungal viruses regulate fungi, etc.). When VLPs are transplanted from one microbiome (i.e., donor) to another (i.e., recipient), the donor VLPs are then able to restructure the recipient's microbiome. As used herein, the term “microbiome” refers all members of the microbiome, including bacteria, viruses, archaea, fungi, and other microscopic eukaryotic organisms.

In the setting of dysbiosis, donor VLPs can regulate the dysbiotic gut microbiome, composition thereby promoting conditions for homeostasis. Existing methods for treating dysbiosis involve using a fecal microbiota filtered to a level that would exclusively retain phages while excluding other VLPs (e.g., US Patent Application Publication 2018/0177831), using fecal phages to treat dysbiosis (e.g., Russian Federation Patent Application Publication 2662310), or using specific phages to target specific bacteria (e.g., US Patent Application Publication 2002/0001590; US Patent Application Publication 2003/0180319).

In contrast, this disclosure describes a system and methods that use an isolated yet diverse population of VLPs to treat dysbiosis, thereby targeting the entire spectrum of microbial residents in the community of the microbiome. However, by restructuring the entire community of microbial residents in a microbiome, a VLP transplantation may incidentally eliminate specific bacteria (e.g., pathogens) that may be detrimental to the homeostasis of the system.

As used here, the VLPs are “isolated” in the sense that they are separated to some degree from non-VLP components of the source material. For example, in some embodiments, the VLPs may be isolated from feces so that the VLP preparation has at least some of the original fecal matter removed. The term “isolated” does not require that the VLPs are completely purified of all non-VLP components of the source material. In some embodiments, however, sanitary concerns may make it desirable that isolated VLPs are substantially free of non-VLP components that could be harmful to a subject to whom the VLP preparation is administered.

Also as used herein, the compositions include a “diverse population” of VLPs that is generally—although need not be precisely—representative of the natural community of VLPs in the source material. Thus, in contrast to existing methods where the community of VLPs is specifically selected to treat particular pathogens, the VLP compositions described herein reflect the broader natural community of VLPs one finds in the source material. Thus, a VLP composition is suitably diverse if it includes a specified percentage of the VLP species natively found in the source material. In some embodiments, the specified percentage of VLP species natively found in the source material to qualify a VLP composition as suitably diverse can be at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 96%, at least 98%, or at least 99%.

The dysbiosis recovery system illustrated in FIG. 1 is exemplified herein using a mouse model, a suitable model for studying dysbiosis. In various alternative embodiments, the systems and methods described herein can be relate to dysbiosis in any mammal of class Mammalia including, but not limited to, a human or nonhuman primate such as a chimpanzee or other ape or monkey species; a farm animal such as cattle, a sheep, a pig, a goat, or a horse; a domestic mammal such as a dog or a cat; a laboratory animal including a rodent such as a mouse, a rat, a guinea pig, or the like. Other suitable hosts for the dysbiosis recovery system include an animal that is, for example, an invertebrate, a bird, an amphibian, a reptile, or a fish. In alternative embodiments, dysbiosis can occur in a plant host including, but not limited to, a plant used in agriculture, botany, horticulture, aquaculture, hydroponics, or aquaponics including, but not limited to, an angiosperm such as wheat, rice, corn, soybean, barley, sorghum, millet, a legume (e.g., pea, bean, lentil, peanut) a daisy, mint, lettuce, tomato, oak, rapeseed, sugar cane, sunflower, potato, cassava, oat, coconut, an oil palm, coffee, banana, sesame, cocoa, beet, apple, plantain, yam, tomato, or the like; or a gymnosperm including, but not limited to, a conifer, a cycad, Ginkgo, or a gnetophyte.

As used herein, “Host in health” will depend on the type of host but is readily known to those of ordinary skill in the art familiar with a particular host. In very general terms, “Host in health” refers to a host that is free from illness, injury, disease, and impairment related to the microbiome.

As used herein, “homeostasis between host and microbiome” refers to a state in which the level of markers of inflammation are within the normal range as used by reference laboratories. Exemplary measures of inflammation in mammals include, but are not limited to, inflammatory markers such as a toll-like receptor (TLR), c-reactive protein, homocysteine, nuclear factor kappa-light-chain-enhancer of activated B cells (NF-κB), interleukin 1 beta (IL-1β), tumor necrosis factor alpha (TNF-α), interferon gamma (IFN-γ), granulocyte-macrophage colony stimulating factor (GM-CSF), interleukin-12 (IL-12), or interleukin-18 (IL-18).

Microbiomes occupy all epithelial surfaces and tissues of animals and plants. The microbiome in the present disclosure is exemplified by the gut microbiome. In alternative embodiments, the dysbiosis recovery system can relate to the microbiome of, for example, the mouth/oral cavity, esophagus, stomach, duodenum, jejunum, ileum, cecum, colon, feces, urinary tract, bladder, vagina, skin, nasal cavity, lung, ear, liver, kidney, heart, spleen, gallbladder, brain, circulatory system, immune system, nervous system, urogenital system, digestive system, musculoskeletal system, or the like. In plants, the dysbiosis recovery system can relate to the microbiome of, for example, the root system, stem system (i.e., leaf, bud, stem, flower, seed, or fruit), and vasculature (i.e., xylem or phloem). In other embodiments, the dysbiosis recovery system can relate to the microbiome of, for example, industrial food production or processing including, but not limited to, a microbiome relating to primary production, postharvest processing, biosanitation, or biodetection relating to, for example, broiler chickens, a packaged meat, a vegetable, a fruit; the fermentation of cheese, yogurt, beer, or wine; or waste treatment including, for example, activated sludge, sewage, or water treatment. In other embodiments, the dysbiosis recovery system can relate to the microbiome of, for example, a medical device or clinical setting such as a stent, a pacemaker, a catheter, a nasogastric tube, an insulin pump, an implant, a prosthetic, a hospitals, a clinic, or an ambulance.

Dysbiosis can be induced in a microbiome by a range of environmental influences. While described above in the context of an exemplary embodiment in which dysbiosis is caused by a high-fat diet, which is typically characterized by a diet high in omega-6 polyunsaturated fatty acids. In alternative embodiments, dysbiosis may be induced by one or more alternative causes including a prebiotic, a probiotic, an antibiotic, a pharmaceutical pharmacological agent, a non-pharmaceutical pharmacological agent, a nutraceutical, a nutritional supplement, a biofilm modifier, a biofilm emulsifier, an autophagy regulator, a phage-encoded protein, fecal transplantation, other microbiota transplantation, a health condition or disorders, a dietary treatment, phage therapy, immunoglobulin therapy, interferon-gamma therapy, growth factor therapy, CRISPR-Cas9, or stem cell transplantation.

Exemplary prebiotics include, for example, a fructo-oligosaccharide, a disaccharide, a monosaccharide, pectin, a polyol, a galacto-oligosaccharide, inulin, a short chain carbohydrate, a sugar alcohol, or oligofructose.

Exemplary probiotics include, for example, Pediococcus, Steptococcus, Lactococcus, Lactobacillus, Oenococcus, or Leuconostoc spp. (e.g., Lactobacillus acidophilus, Lactobacillus plantarum, Lactobacillus casei, Lactobacillus paracasei, Lactobacillus delbrueckii, Lactobacillus brevis, Lactobacillus fermentum, Lactobacillus johnsonii, Lactobacillus salivarius, Lactobacillus sakei, Lactobacillus bulgarius, Lactobacillus reuteri, Lactobacillus rhamnosus GG, Lactobacterium lactis, Leuconostoc mesenteroides), Streptococcus thermophilus, Saccharomyces boulardii, Saccharomyces bayanus, Saccharomyces cerevisiae, Bifidobacterium bifidum, Bifidobacterium infantis, Bifidobacterium adolescentis, Bifidobacterium longum, Bifidobacterium breve, Bifidobacterium lactis, Bifidobacterium animalis, Bacillus coagulans, Bacillus subtilis, Bacillus cereus, Pedicoccus acidilactici, Escherichia coli Nissle, Enterococcus durans, or Enterococcus faecium.

Exemplary antibiotics include, for example, vancomycin, amoxicillin, metronidazole, cephalexin, trimethoprim/sulfamethoxazole, doxycyline, ciprofloxacin, colistin, tetracycline, cephalexin, levofloxacin, moxifloxacin, ofloxacin, norfloxacin, ampicillin, erythromycin, tinidazole, nitazoxanide, albendazole, paromomycin, quinacrine, lactulose, bismuth subsalicylate, gentamicin, neomycin, ceftobiprole, ceftaroline, clindamycin, dalbavancin, fusidic acid, linezolid, mupirocin, omadacycline, oritavancin, tedizolid, telavancin, tigecycline, an aminoglycoside, a carbapenem, ceftazidime, cefepime, ceftolozane/tazobactam, a fluoroquinolone, piperacillin/tazobactam, ticarcillin/clavulanic acid, a streptogramin, or daptomycin.

Exemplary pharmaceuticals and non-pharmaceutical pharmacological agents include, for example, an antipyretic, an analgesic, an antimalarial drug, an antibiotic, an antiseptic, a mood stabilizer, a hormone replacement, an oral contraceptive, a stimulant, a tranquilizer, a statin, an antacid, a reflux suppressant, an antiflatulent, an antidopaminergic, a proton pump inhibitor, an H2-receptor antagonist, a cytoprotectant, a laxative, an antidiarrheal, a bile acid sequestrant, an opioid, a calcium channel blocker, a diuretic (e.g., a thiazide diuretic, a loop diuretic,), a cardiac glycoside, an antiarrhythmics, a nitrate, an antianginal, a vasoconstrictor, a vasodilator, an ACE inhibitor, an angiotensin receptor blocker, a beta-blocker, an a blocker, an aldosterone inhibitor, an anticoagulant, heparin, an antiplatelet drug, a fibrinolytic, an antifibrinolytic, an anti-hemophilic factor, a haemostatic drug, a hypolipidaemic agent, an NSAID, a local anesthetic, a benzodiazapine, a barbiturate, a neuromuscular drug, am anticholinesterase, an adrenergic neuron blocker, an astringent, an ocular lubricant, a topical anesthetic, a sympathomimetic, a parasympatholytic, a mydriatic, a cycloplegic, an antibiotic, a topical antibiotic, a sulfa drug, an aminoglycoside, a fluoroquinolone, an antiviral, an imidazole, a polyene, a corticosteroid, a mast cell inhibitor, mast cell stabilizer, an adrenergic agonist, a carbonic anhydrase inhibitor/hyperosmotic, a cholinergic, a miotic, a parasympathomimetic, a prostaglandin, a prostaglandin agonist, a prostaglandin inhibitor, a prostaglandin analog, nitroglycerin, a sympathomimetic, an antihistamine, an anticholinergic, an antifungal, a cerumenolytic, a bronchodilator, an antitussive, a mucolytic, a decongestant, a leukotriene antagonist, an androgen, an antiandrogen, an estrogen, gonadotropin, human growth hormone, an antidiabetic (e.g., a sulfonylurea, a biguanide/metformin, a thiazolidinedione, insulin), a thyroid hormone, an antithyroid drug, calcitonin, diphosponate, a vasopressin analog, an alkalinizing agent, a quinolone, an anticholinergic, an antispasmodic, 5-alpha reductase inhibitor, a selective alpha-1 blocker, a sildenafil, a fertility medication, hormonal contraception, ormeloxifene, a spermicide, Hormone Replacement Therapy (HRT), a bone regulator, beta-receptor agonists, follicle stimulating hormone, luteinizing hormone, LHRH, gamolenic acid, gonadotropin release inhibitor, progestogen, a dopamine agonist, an estrogen, gonadorelin, clomiphene, tamoxifen, diethylstilbestrol, an emollient, an anti-pruritic, a disinfectant, a scabicide, a pediculicide, a tar product, a vitamin A derivative, a vitamin D analog, a keratolytic, an abrasive, a hormone, a desloughing agent, an exudate absorbent, a proteolytis, a sunscreen, an antiperspirant, an immune modulator, an antileprotic, an antituberculous drug, an anthelmintic, an amoebicids, an antiprotozoal, an antitoxin, an antivenom, a vaccine, an immunoglobulin, an immunosuppressant, an interferon, a monoclonal antibody, a tonic, an electrolyte and/or mineral preparation (e.g., an iron preparation and/or a magnesium preparation), parenteral nutrition, a vitamin, an anti-obesity drug, an anabolic drug, a haematopoietic drug, a food product drug, a cytotoxic drug, a therapeutic antibody, a sex hormone, an aromatase inhibitor, a somatostatin inhibitor, a recombinant interleukin, G-CSF, erythropoietin, a euthanaticum, acetaminophen, dextroamphetmine-amphetamine (e.g., ADDERALL, Richwood Pharmaceutical Co., Inc., Florence, Ky.), alprazolam (e.g., XANAX, Pharmacia & Upjohn Co., LLC, Kalamazoo, Mich.), amitriptyline, amlodipine, atorvastatin, azithromycin, citalopram, clonazepam, cyclobenzaprine, duloxetine (e.g., CYMBALTA, Eli Lilly and Co., Indianapolis, Ind.), doxycycline, gabapentin, hydrochlorothiazide, ibuprofen, escitalopram (e.g., LEXAPRO, Forest Laboratories, Inc., New York, N.Y.), lisinopril, loratadine, lorazepam (e.g., ATIVAN, American Home Products Corp., New Yor.k, N.Y.), losartan, meloxicam, metformin, metoprolol, naproxen, omeprazole, pantoprazole, prednisone, pregabalin (e.g., LYRICA, Parke, Davis & Co., LLC, Morris Plains, N.J.), risperidone, tramadol, trazodone, sildenafil (e.g., VIAGRA, Pfizer, Inc., New York, N.Y.), bupropion (e.g., WELLBUTRIN, GlaxoSmithKline LLC, Wilmington, Del.), sertraline (e.g., ZOLOFT, Pfizer, Inc., New York, N.Y.), adalimumab, ledipasvir/sofosbuvir, etanercept, infliximab, rituximab, insulin glargine, bevacizumab, trastuzumab, lenalidomide, fluticasone proprionate, rosuvastatin, amphetamines, marijuana/cannabis (tetrahydrocannabinol [THC]), cocaine or a metabolite of cocaine, psilocybin, an opiate (e.g., heroin, morphine, codeine), a synthetic opioid (e.g., oxycodone, hydrocodone, buprenorphine, or methadone) other scheduled substances.

Exemplary nutraceuticals and nutritional supplements include, for example, prenatal vitamins, folic acid, garcinia cambogia, a raspberry ketone, a green tea supplement, turmeric, curcumin, coenzyme q-10, cranberry, fish oil, garlic, ginger, ginkgo, ginseng, a collagen peptide, s-adenosylmethionine, creatine, MCT oil, a branched amino acid, L-arginine, elderberry, activated charcoal, psyllium husk powder, maca, orlistat (e.g., ALLI diet pills, GlaxoSmithKline LLC, Wilmington, Del.), chia, a superfood, milk thistle, apple cider vinegar, hemp oil, cannabidiol (CBD), chromium, chondroitin sulfate, cassia cinnamon, calcium, glucosamine, green tea, hoodia, magnesium, melatonin, saw palmetto, selenium, St. John's wort, valerian, ascorbic acid, zinc, echinacea, a probiotic, an omega fatty acid, an alpha-lipoid acid, a retinoid, carotene, thiamin, riboflavin, niacin, pantothenic acid, pyridoxine, cobalamin, biotin, choline, calciferol, alpha-tocopherol, phylloquinone, menadione, a vitamins, or a nutritional mineral.

Exemplary biofilm modifiers and emulsifiers include, for example, egg lecithin, soy lecithin, calcium stearoyl-2-lactylate (CSL), polyglycerol ester (PGE), guar gum, carrageenan, a deoxyribonuclease, N-acetylcysteine, a Salvadora persica extract, a cranberry component, a cathelicidin-derived peptide, lactoferrin, a synthetic iron chelator, competence-stimulating peptide, an alginate lyase, ozone, xylitol hydrogel, S-adenosylmethionine, S-adenosyl-homocysteine, sinefungin, an N-sulfonyl homoserine lactone, glycoside hydrolase dispersin B, curcumin, a Delisea pulchra furanone, a ribonucleic acid III inhibiting peptide, a polysorbate, a monoglyceride, a diglyceride, nitric oxide, acetyl-11-keto-β-boswellic acid, patulin, penicillic acid, a small lytic peptide, PTP-7, a phage, or a silver nanoparticle.

Exemplary autophagy regulators include, for example, metformin, perifosine, rapamycin, everolimus, perveratrol, MG-132, trichostatin A, suberoylanilide hydroxamine (vorinostat), Z-VAD-FMK, zinc, vitamin D, alkaline phosphatase, CCl-779, glucose, glucose-6-phosphate, lithium, L-690, L-330, carbamazepine, sodium valproate, verapamil, loperamide, amiodarone, nimodipine, nitrendipine, niguldipine, pimozide, calpastatin, calpeptin, clonidine, rilmenidine, 2′5′-dideoxyadenosine, NF449, minoxidil, penitrem A, fluspiriline, trifluoperazine, trehalose, SMER10, SMER18, or SMER28.

Exemplary phage-encoded proteins and genes include, for example, an endolysin, a holin, an artilysin, a capsid protein, a tail protein, an antibiotic resistance gene, an antibiotic susceptibility gene, an antioxidation gene, an oxidative stress gene, a metabolic gene, a biofilm gene, an adherence gene, shiga toxin, diphtheria toxin, botulinum toxin, an adherence protein, or another virulence factor.

Exemplary fecal microbiota transplants (FMTs) or other microbiota transplants include, for example, whole fecal material, partial fecal material, altered fecal material, whole microbiota, partial microbiota, or altered microbiota. As used herein, “whole fecal material” refers to the excretory product from the bowel. As used herein, “partial fecal material” refers to one or more components of the excretory product from the bowel. As used herein, “an altered fecal material” refers to an excretory product from the bowel that is treated to remove, add, or change the function or property of one or more components. As used herein, “whole microbiota” refers to the entirety of the community of microorganisms. As used herein, “partial microbiota” refers to one or more species of a community of microorganisms. As used herein, “altered microbiota” refers to a community of microorganisms that is treated to remove, add, or change the function or property of one or more species of microorganisms.

Exemplary alternative physical or psychological disorders include, for example, physical or psychological stress, Crohn's Disease, ulcerative colitis or other inflammatory bowel disease, irritable bowel syndrome (IBS), small intestinal bacterial overgrowth, bloating, flatulence, constipation, diarrhea, infection associated with malaria, Chagas disease, giardiasis, filariasis, toxoplasmosis, schistosomiasis, amoebiasis, onchocerciasis, ascariasis, trichinosis, pinworm infection, babesiosis, trichuriasis, head lice, fasciolosis, clonorchiasis, gnathostomiasis, influenza, isosporiasis, fasciolopsiasis, diphyllobothriasis, hymenolepiasis, lyme disease, jock itch, athlete's foot, impetigo, aspergillosis, blastomycosis, candidiasis, coccidioidomycosis, histoplasmosis, fungal eye infections, fungal nail infections, mucormycosis, mycetoma, pneumocystis pneumonia, sporotrichosis, talaromycosis, dermatomycosis, sepsis, functional abdominal pain and other functional disorders, chronic fatigue syndrome, fibromyalgia, interstitial cystitis, functional abdominal pain, chronic pelvic pain syndrome, cirrhosis, end stage renal disease, celiac disease, gluten sensitivity, gluten intolerance, an allergy (e.g., a food allergy), asthma, diabetes, metabolic syndrome, cardiovascular disease, Gulf War Syndrome or Illness, leaky gut syndrome, cystic fibrosis, multi-symptom disorder, a hypersensitivity disorder, multiple food or chemical sensitivity, environmental illness, food sensitivity, food intolerance, halitosis, sugar craving, autism, attention deficit hyperactivity disorder (ADHD), post-traumatic depression, post-traumatic stress disorder, anxiety, multiple sclerosis, neuropathy, fogginess of head, brain fog, impaired mentation, difficulty with concentration, impaired memory, Alzheimer's disease, dementia, mild cognitive impairment, restless leg syndrome, moodiness, irritability, sleep disorders, headaches, migraines, systemic lupus erythromatosis, scleroderma, rheumatoid arthritis, osteoarthritis and other autoimmune disorders, Hashimoto's thyroiditis, atopic dermatitis, eczema, psoriasis, acne, urticaria, rosacea, tinea versicolor, tinea corporis, asthma, atherosclerosis or a related cardiovascular disease, vaginosis, periodontal disease, gingivitis, dental caries, disease of pulp chamber of a tooth, iritis, uveitis, or obesity. Additional exemplary disorders include microbial infections such as, for example, infection by Acinetobacter baumannii, Bacillus anthracis, Bacillus cereus, Bacteroides fragilis, Burkholderia cepacian, Campylobacter jejuni, Clostridium difficile, Clostridium sordellii, Cronobacter sakazakii, an Enterobacteriaceae spp. (including carbapenem-resistant Enterobacteriaceae), Enterococcus faecalis, Escherichia coli (e.g., O157:H7), Hepatitis A, Hepatitis B, Hepatitis C, human immunodeficiency virus (HIV), Kliebsiella pneumoniae, Staphylococcus aureus (including methicillin-resistant or vancomycin-resistant S. aureus), Listeria monocytogenes, Mycobacterium smegmatis, Morganella morganii, Mycobacterium abscessus, Norovirus, Psuedomonas aeruginosa, Stenotrophomonas maltophilia, Mycobacterium tuberculosis, Enterococci (including vancomycin-resistant Enterococci), a Salmonella spp., a Brucella spp., a Shigella spp., Ebolavirus, a Lyssavirus (e.g., rabies virus), Candida auris, Cryptococcus neoformans, Cryptococcus gattii, or Zika virus.

Exemplary alternative dietary treatments include, for example, low fiber, high sugar, ketogenic, paleo, Mediterranean, Atkins, fermented, vegetarian, vegan, raw food, South Beach, zone, blood type, DASH, Whole30, MIND, fertility, TLC, Weight Watchers, Mayo Clinic, volumetrics, flexitarian, Jenny Craig, Biggest Loser, Ornish, Slim Fast, SparkPeople, Anti-Inflammatory, HMR, Flat belly, Nutrisystem, Engine 2, Abs, Eco-Atkins, Glycemic-Index, macrobiotic, Medifast, supercharged hormone, acid alkaline, fast, body reset, or dukan.

A perturbed microbiome during dysbiosis has numerous consequences for the host. Dysbiosis can be characterized in many ways and is either identified by examining either the microbiome or the host. When examining the microbiome, dysbiosis can be detected by assessing the diversity of the gut bacterial community. Diversity during dysbiosis is decreased and certain bacterial members expand to make up a larger proportion of the total population. For example, during gut microbiome dysbiosis, the phylum Firmicutes expands relative to the phylum Bacteroidetes. Dysbiosis can therefore be diagnosed by looking at the ratio of these two phyla. An increased Firmicutes-Bacteroidetes ratio is a common marker of dysbiosis resulting from an HFD or a Western diet (FIG. 2, HFD-PBS). Other forms of dysbiosis are associated with a decreased Firmicutes-Bacteroidetes ratio. An increase or decrease in the Firmicutes-Bacteroidetes ratio in a subject is typically measured against a suitable control. A suitable control may be, for example, the Firmicutes-Bacteroidetes ratio observed in the subject when known not to be experiencing dysbiosis. Alternatively, a suitable control may be the Firmicutes-Bacteroidetes ratio from an individual or group of individuals known not to be experiencing dysbiosis. As another example, a suitable control Firmicutes-Bacteroidetes ratio may be an art-recognized standard.

Additionally, dysbiosis can be characterized by unregulated growth of the bacterial population as a whole. In the setting of gut microbiome dysbiosis, overgrowth of bacteria into the otherwise sterile small intestine, a disorder called small intestinal bacterial overgrowth (SIBO), is a common marker of dysbiosis (FIG. 3, HFD-PBS). Small intestinal bacterial overgrowth (SIBO) is a form of dysbiosis affecting many patients with IBS. It is a disorder of the gastrointestinal tract characterized by increased bacterial density in the small intestine. Bacterial overgrowth is dysbiosis characterized by increased bacterial density anywhere on the epithelial surface of an organ. In the gastrointestinal tract, a healthy small intestine is virtually sterile, particularly in the proximal end of the organ. The proliferation of bacteria into this region is associated with heightened immune response, leaky gut, and increased translocation of bacteria across the gut barrier. SIBO is most associated with IBS but also has associations with Crohn's Disease, chronic fatigue syndrome, fibromyalgia, anxiety and depression. Currently, treatment regimens for SIBO are limited to administering antibiotics such as rifaximin or neomycin.

Dysbiosis is ultimately a change in the composition of the microbiome. The compositional change promotes pathogenic behavior of the microbiome's members. The overall composition of the microbiome may be assessed by comparing the beta diversity of a subject's microbiome to a healthy microbiome standard. Beta diversity is determined using 16 s rRNA sequencing. Briefly, 16 s rRNA sequencing analyzes all of the members of one community (e.g., bacterial, viral, or fungal) and assesses their similarity to all of the members of another community. The complexity of each community can be simplified into a single data point that is placed on a principal coordinates analysis plot. Similarity between communities can be determined based on how the points cluster on the plot. A high-fat diet alters the beta diversity of a microbiome (FIG. 4, HFD-PBS) so that it clusters separately from a healthy microbiome (FIG. 4, SD-PBS), demonstrating dissimilarity and dysbiosis. In addition to beta diversity, the composition of the metagenome, the transcriptome, and/or the metabolome also add to the pathogenic potential of the microbiome that contributes to dysbiosis. For example, presence of genes for shiga toxin in the metagenome can make otherwise harmless resident gut bacteria become pathogenic.

While described above in the context of an exemplary embodiment in which dysbiosis is characterized by an increased Firmicutes-Bacteroidetes ratio (FIG. 2, HFD-PBS), increased bacterial density in the small intestine (small intestinal bacterial overgrowth [SIBO]) (FIG. 3, HFD-PBS), and an altered beta diversity (FIG. 4, HFD-PBS), the compositions and methods described herein can involve treating or restoring homeostasis from dysbiosis characterized in any suitable manner. Exemplary methods for detecting dysbiosis include, for example, 16s r RNA sequencing, pyrosequencing, metagenomic sequencing, shotgun sequencing, biopsy, complete blood count, basic metabolic panel, antinuclear antibody panel, c-reactive protein test, homocysteine test, erythrocyte sedimentation rate (ESR), a blood enzyme test, lipoprotein panel, urine test, polymerase chain reaction, enzyme-linked immunosorbent assay, immunohistochemistry, western blot, immunocytochemistry, immunofluorescence, immunoassay, flow cytometry, fluorescent labeling, high performance liquid chromatography (HPLC), Mass spectrometry, fluorescent microscopy, tangential flow filtration, and electron microscopy.

The methods described above may be used to identify one or more characteristics of dysbiosis. Exemplary characteristics of dysbiosis within the microbiological community include, for example, increased Proteobacteria, decreased Proteobacteria, expansion of sulfate-reducing bacteria species, increased hydrogen sulfide in the circulation, increased Verrucomicrobia, decreased Verrucomicrobia, increased microbiome diversity, richness, or evenness, decreased microbome diversity, richness, or evenness, increased metabolome diversity, richness, or evenness, decreased metabolome diversity, richness, or evenness, increased transcriptome diversity, richness, or evenness, decreased transcriptome diversity, richness, or evenness, increased metagenome diversity, richness, or evenness, decreased metagenome diversity, richness, or evenness, increased virome diversity, richness, or evenness, decreased virome diversity, richness, or evenness, expansion of Caudovirales, depletion of Caudovirales, expansion of Microviridae, depletion of Microviridae, expansion of extracellular VLPs, expansion of temperate or lysogenic phages, depletion of temperate or lysogenic phages, fungal infection, bacterial infection, or viral infection. In each of the above cases, an increase or decrease is measured relative to a comparison group of individuals that serve as a control group.

When identifying impaired epithelial function in the gut, one can assess markers in the host such as alterations in the host's epithelial mucus layer, expression of inflammatory markers, metagenome, transcriptome, and metabolome. The Muc2 protein is an extracellular mucin protein that makes up the epithelial mucus layer and is released from goblet cells in the intestinal epithelium. Downregulation of Muc2 expression indicates an impaired mucus layer (FIG. 4, HFD-PBS). The mucus layer is a primary defense against the inflammatory contents of the intestinal lumen. An impaired mucus layer is a condition that promotes heightened inflammation and leads to disease states associated with dysbiosis.

While described above in the context of an exemplary embodiment in which dysbiosis is characterized by decreased expression of mucin protein Muc2 (FIG. 5, HFD-PBS) and/or SIBO, the compositions and methods described herein can involve restoring homeostasis and/or treating dysbiosis associated with a medical condition. Exemplary dysbiosis-related conditions include, for example, Crohn's Disease, ulcerative Colitis, an inflammatory bowel disease, irritable bowel syndrome (IBS), small intestinal bacterial overgrowth (SIBO), chronic fatigue syndrome, fibromyalgia, interstitial cystitis, chronic pelvic pain syndrome, cirrhosis, end stage renal disease, celiac disease, gluten sensitivity, gluten intolerance, allergy, asthma, diabetes, metabolic syndrome, cardiovascular disease, Gulf War Syndrome or Illness, leaky gut syndrome, cystic fibrosis, multi-symptom disorder, a hypersensitivity disorder, halitosis, autism, attention deficit hyperactivity disorder (ADHD), post-traumatic depression, post-traumatic stress disorder, multiple sclerosis, Alzheimer's disease, restless leg syndrome, a sleep disorder, systemic lupus erythematosus, scleroderma, rheumatoid arthritis, osteoarthritis, an autoimmune disorder, Hashimoto's thyroiditis, atopic dermatitis, eczema, psoriasis, acne, urticaria, rosacea, atherosclerosis, vaginosis, periodontal disease, gingivitis, dental caries, disease of pulp chamber of a tooth, iritis uveitis, or obesity.

Exemplary clinical signs and symptoms of such conditions include, for example: bloating, flatulence, constipation, diarrhea, functional abdominal pain and other functional disorders, functional abdominal pain, disrupted metagenome, disrupted transcriptome, disrupted metabolome, sugar craving, anxiety, neuropathy, fogginess of head, brain fog, impaired mentation, difficulty with concentration, impaired memory, dementia, mild cognitive impairment, moodiness, irritability, headaches, migraines, irregular epithelial tissue morphology, loose stool consistency, bowel movement irregularity, increased blood sugar, decreased blood sugar, increased blood pressure, decreased blood pressure, increased heart rate, decreased heart rate, or increased or decreased expression of inflammatory markers (e.g., c-reactive protein, fibrinogen, interleukins, MMP-9, CD14, cytokines, and corticosteroids, antimicrobial peptides, heat shock proteins, or autophagy genes).

This disclosure describes systems and methods for treating dysbiosis. Existing treatments for dysbiosis include antibiotics, such as rifaximin, or fecal microbiota transplant (FMT). These treatments are not always effective and recurrence of dysbiosis is common. In contrast, the approach for treating dysbiosis described herein involves virus-like particle (VLP) transplantation. VLP transplantation uses a population of VLPs that has been isolated from a donor microbiota (e.g., the fecal microbiota) for transplantation into a recipient microbiota. The collection of viruses that make up a VLP population are collectively infectious to the breadth of cellular hosts in a recipient microbiome, giving it a natural advantage in modifying host-associated microbiomes. When the VLPs are isolated from the other components of the microbiota (i.e., bacteria, archaea, fungi, organic matter, etc.), they are uninhibited by donor-derived, non-VLP fecal components. This allows for the VLPs to have more direct interactions with all of the diverse resident cellular microorganisms in a recipient microbiome. This gives VLP transplantation a higher potential for altering all members of the resident microbial community.

Each individual component of the VLP population has unique qualities that can be leveraged for the treatment of dysbiosis. In general, VLPs can regulate the overall bacterial population density, increasing diversity of the bacterial community, transfer genes among bacterial members or other cells in the host epithelium, maintain intestinal homeostasis (e.g., healthy mucus layer and intestinal barrier function), and/or promote mucosal immunity.

Phages (bacteria-specific viruses) are a large proportion of the VLP community. Historically, individual phage species have been isolated from the environment for use in phage therapy. Phage therapy is the practice of propagating environmental phages to high titers for treatment of bacterial infections. Phages are host specific and only infect a narrow range of bacteria. Phage therapy has been successful in treating numerous types of bacterial infections including those of the skin, the gastrointestinal tract, and the bloodstream. Phages also are highly effective against antibiotic resistant bacteria or multidrug resistant organisms. With the growing problem of resistance to antibiotics, there is immense potential for phage therapy as an alternative therapy when treating bacterial infections, which typically involves a small number of bacterial pathogens.

A different strategy is needed to treat microbiome dysbiosis, which is a disorder that involves the entire microbial community (i.e., eukaryotes, bacteria, archaea, fungi, viruses, and phages), the metagenome, transcriptome, and metabolome. The dysregulation of the entire microbiome during dysbiosis can cause systemwide consequences to the host. This disclosure describes VLP transplantation, a novel therapy for treating dysbiosis using VLPs, including phages, eukaryotic viruses, archaeal viruses, and/or gene transfer agents (GTAs). VLP transplantation is effective since each individual component of the VLP community corresponds with a cellular member of the microbiome (i.e., fungal viruses infect fungi; archaeal viruses infect archaea). Because VLPs can infect and regulate their respective cellular hosts, treatment with a diverse VLP population has an advantage because it can target the entire microbiome membership. VLP transplantation therefore leverages the strategy of phage therapy for all microorganisms in a microbiome. VLPs can be found in any environment with mixed microbial community making them abundant and easily obtained.

In some embodiments, a VLP composition involves preparing a VLP transplant using an isolation step exemplified by, but not limited to, centrifugation, ultracentrifugation, filtration, size fractionation, affinity purification, affinity chromatography, high performance liquid chromatography (HPLC), a CsCl or other density gradient, tangential flow filtration, polyethylene glycol treatment, chloroform treatment, chemical flocculation, and/or entrapment of VLPs into polymers.

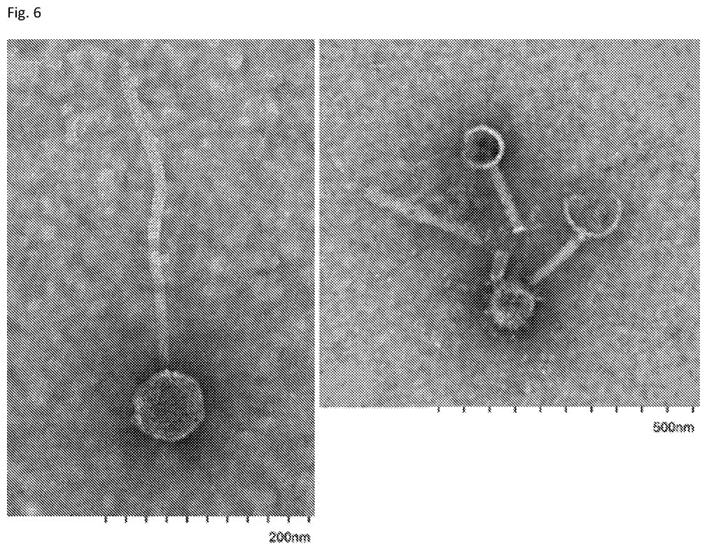

VLPs regulate both prokaryotic and eukaryotic populations in their environment, affecting cell bacterial growth, competitive ability, and, in the case of a microbiome, interactions with the associated host. For example, VLPs were collected and enriched from the feces of a donor mouse. The enrichment process involved homogenization of the fecal sample in phosphate-buffered saline (PBS), agitation of the solution using 1.0-mm zirconium silicate beads to disaggregate VLPs from particles, centrifugation to pellet bacterial cells, eukaryotic cells, and other large particles, and filtration at 0.45 microns to further remove bacteria. The final VLP enrichment step involved capturing VLPs on a 100 kDa centrifugal filter membrane (equivalent to a 3 nm pore size) where they were washed successively with PBS before being resuspended in PBS. Presence of VLPs in resuspension was confirmed using electron microscopy (FIG. 6). Isolated VLPs were administered in a mouse model of a high-fat diet (HFD)-induced dysbiosis. Host-microbiome dysbiosis was demonstrated by an increased Firmicutes-Bacteroidetes ratio (FIG. 2, HFD-PBS), and increased bacterial density (FIG. 3, HFD-PBS) and an altered beta diversity (FIG. 4, HFD-PBS). VLPs also reduced signs of disease in the host such as downregulated Muc2 expression (FIG. 5, HFD-PBS) and helped restore regulation of the resident gut bacterial population by reducing bacterial overgrowth (FIG. 3, HFD).

VLP transplantation ameliorated characteristics of dysbiosis such as increased Firmicutes-Bacteroidetes ratio (FIG. 2). The ratio of the phyla Firmicutes and Bacteroidetes in the ileal microbiome of the HFD-PBS group was on average 3.2-times higher than in the SD-PBS Control group (HFD-PBS=6.3±5.7; SD-PBS=2.0±3.6) (p<0.05). Treatment of HFD animals with VLPs reduced the Firmicutes-Bacteroidetes ratio to a level (FIG.2 HFD-VLP) similar to that of Controls (FIG. 2, SD-PBS). Specifically, the Firmicutes-Bacteroidetes ratio of the HFD-VLP group (1.2±0.9) was not significantly different from the Control group (p=0.99).

VLP transplantation ameliorated additional characteristics of dysbiosis in the ileum such as bacterial overgrowth (FIG. 3). The HFD-PBS group had on average a 3.2-fold increase in bacterial density relative to the SD-PBS Control group (HFD-PBS=3.2±1.0; SD-PBS=1.1±0.2) (p<0.05). Treatment of HFD animals with VLPs reduced the bacterial density (FIG.3, HFD-VLP) to a level similar to that of Controls (FIG. 3, SD-PBS). Specifically, the bacterial density of the HFD-VLP group (0.9±0.4) was significantly less than the HFD-PBS group (p<0.05) but was not significantly different from the SD-PBS Control group (p=0.8). This demonstrates the utility of VLP transplantation for reducing bacterial overgrowth in the microbiome while simultaneously restoring the host's ability to defend against bacterial overgrowth, thereby restoring homeostasis.

VLP transplantation ameliorated signs of disease in the host such as reduced Muc2 expression in the small intestinal ileum (FIG. 5). The HFD-PBS group had on average 0.3-times the Muc2 expression relative to the SD-PBS Control group (HFD-PBS=0.3±0.04; SD-PBS=1.1±0.3) (p<0.05). Treatment of HFD animals with VLPs increased Muc2 expression (FIG. 5, HFD-VLP) to a level similar to that of Controls (FIG. 5, SD-PBS). Specifically, the relative Muc2 expression of the HFD-VLP group (1.0±0.1) was significantly more than the HFD-PBS group (p<0.05) but was not significantly different from the SD-PBS Control group (p=0.99).

These results support the conclusion that treatment with a VLP population at the site of dysbiosis in a host-associated microbiome is sufficient to restore homeostasis. In the present example, mice with HFD-induced gut dysbiosis were treated with VLPs, which restored Muc2 expression, bacterial population density, and the Firmicutes-Bacteroidetes ratio in the ileum to the level of healthy, control mice. This finding on the resolution of dysbiosis is the first evidence of a VLP-mediated therapy for dysbiosis.

Additionally, treatment of mice with an HFD altered the beta diversity (i.e., bacterial community composition) of the ileal microbiome (FIG. 4, HFD-PBS), demonstrating dysbiosis in HFD-treated mice. This was determined through analysis of the weighted UniFrac distances from the 16 s rRNA sequencing results showing that clustering of the HFD-PBS group was significantly different from the SD-PBS Control group (p<0.01) on the principal coordinates analysis plot. SD mice treated with VLPs from an HFD donor were significantly different from the SD-PBS Control group (p<0.05) (FIG. 4, SD-VLP) and had a community composition associated with the HFD-PBS cluster.

These results support the conclusion that a VLP transplantation can be used to transfer a donor microbiome composition into a recipient. This can include altering the microbiome, metagenome, transcriptome, and/or metabolome in a recipient to resemble the donor. This finding on the VLP-mediated transfer of microbiome, metagenome, transcriptome, and metabolome composition is the first evidence of a VLP-based transfer of microbiomes, metagenome, transcriptome, and/or metabolome between individuals.

Most commonly, conventional phage therapy involves the use of a single phage targeting a single bacterial strain or a “phage cocktail,” which is an artificial preparation of a limited, small subset of phage species found to target specific bacteria of interest. This approach is limited by the ability of the phages chosen to target the specific strain causing infection and the ability of the bacteria to evolve resistance to phages (analogous to bacteria evolving resistance to antibiotics), ultimately making the phage cocktail ineffective. In contrast, the VLP preparations described herein, and methods that involve administering such VLP preparations, exploit a strategy to overcome the problem of bacterial resistance. The VLP preparation includes a heterogeneous, highly diverse gut phage population. The high diversity in the heterogenous VLP preparation can overwhelm target bacteria that are incapable of evolving effective resistance to multiple infective VLPs and may also be useful in overcoming multi-drug resistant bacterial pathogens. The diverse phages may lead to increased bacterial diversity through mechanisms such as “Kill the Winner” dynamics, whereby phages capable of infecting common bacteria will be amplified due to large densities of hosts, thus driving density of this particular population down. As such, even when the VLPs are not targeting a particular bacterium, they can reduce its ability to invade the host environment through their effects on microbiome diversity. Leveraging this broad community of phages thus provides an alternative strategy to conventional phage therapy.

Thus, this disclosure describes VLP preparations and the use of VLP preparations for the treatment of conditions caused by dysbiosis. As used herein, “treat” or variations thereof refer to reducing, limiting progression, ameliorating, or resolving, to any extent, the symptoms or signs related to a condition. A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is initiated before a condition manifests in a subject.

Treatment that is prophylactic—e.g., initiated before a subject manifests a symptom or clinical sign of the condition such as, for example, while an infection remains subclinical—is referred to herein as treatment of a subject that is “at risk” of having the condition. As used herein, the term “at risk” refers to a subject that may or may not actually possess the described risk. Thus, for example, a subject “at risk” of infectious condition is a subject present in an area where other individuals have been identified as having the infectious condition and/or is likely to be exposed to the infectious agent even if the subject has not yet manifested any detectable indication of infection by the microbe and regardless of whether the subject may harbor a subclinical amount of the microbe. As another example, a subject “at risk” of a non-infectious condition is a subject possessing one or more risk factors associated with the condition such as, for example, genetic predisposition, ancestry, age, sex, geographical location, lifestyle, or medical history. As yet another example, a subject who had been treated for dysbiosis is “at risk” for a recurrence or relapse of dysbiosis. Prophylactic treatment may be initiated before a subject manifests symptoms or clinical signs of the condition or when a subject manifests prodromal symptoms or clinical signs that are harbingers of a full-blown recurrence or relapse of dysbiosis. Such prophylactic treatment may be directed at reducing the likelihood and/or severity of any recurrence or relapse or reduce of dysbiosis. For example, a fluctuating energy level may be a prodromal symptom of an impending recurrence or relapse of dysbiosis in chronic fatigue syndrome.

Accordingly, a VLP preparation may be administered to a subject before, during, or after the subject first exhibits a symptom or clinical sign of the condition caused by dysbiosis. Treatment initiated before the subject first exhibits a symptom or clinical sign associated with the condition may result in decreasing the likelihood that the subject experiences clinical evidence of the condition compared to a subject to which the VLP preparation is not administered, decreasing the severity of symptoms and/or clinical signs of the condition, and/or completely resolving the condition. Treatment initiated after the subject first exhibits a symptom or clinical sign associated with the condition may result in decreasing the severity of symptoms and/or clinical signs of the condition compared to a subject to which the VLP preparation is not administered, and/or completely resolving the condition.

Thus, the method includes administering an effective amount of the composition to a subject having, or at risk of having, a condition caused by dysbiosis. In this aspect, an “effective amount” is an amount effective to reduce, limit progression, ameliorate, or resolve, to any extent, a symptom or clinical sign related to the condition.

The VLP preparation described herein may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic VLP preparations is contemplated. Supplementary active ingredients also can be incorporated into the VLP preparations. As used herein, “pharmaceutically acceptable” refers to a material that is not biologically or otherwise undesirable, i.e., the material may be administered to an individual along with VLP preparation without causing any undesirable biological effects or interacting in a deleterious manner with any of the other components of the pharmaceutical VLP preparation in which it is contained.

The VLP preparation may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), via a tube placed in an organ such as the gastrointestinal tract or a body cavity, or topical (e.g., intranasal, intrapulmonary, intramammary, intravaginal, intrauterine, intradermal, transcutaneous, rectally, intracystic, etc.). A pharmaceutical composition can be administered to a mucosal surface, such as by administration to, for example, the nasal or respiratory mucosa (e.g., by spray or aerosol). A composition also can be administered via a sustained or delayed release.

Thus, the VLP preparation may be provided in any suitable form including but not limited to a solution, a suspension, an emulsion, a spray, an aerosol, or any form of mixture. The VLP preparation may be delivered in formulation with any pharmaceutically acceptable excipient, carrier, or vehicle. For example, the formulation may be delivered in a conventional topical dosage form such as, for example, a cream, an ointment, a skin patch, an aerosol formulation, a non-aerosol spray, a gel, a lotion, a hand wash, a body wash, a shampoo, a surgical or dental wash or rinse, a dentrifice, an eye drop, an inhaler, and the like. The formulation may further include one or more additives including such as, for example, an adjuvant, a skin penetration enhancer, a colorant, a fragrance, a flavoring, a moisturizer, a thickener, and the like.

In some cases, the method of treatment may involve treating dysbiosis localized to an epithelial tissue such as, for example, the epidermis, a mucosal surface, at least a portion of the skin or scalp, at least a portion of the oral cavity including tooth or gum, at least a portion of the gastrointestinal tract, at least a portion of the nasal cavity, at least a portion of the respiratory tract, at least a portion of the genito-urinary tract, or a body cavity. Accordingly, the VLP preparation may be provided in a formulation suitable for delivery to an epithelial tissue by a route of administration suitable for delivery to the epithelial tissue.

A formulation may be conveniently presented in unit dosage form and may be prepared by methods well known in the art of pharmacy. Methods of preparing a pharmaceutical composition with a pharmaceutically acceptable carrier include the step of bringing the VLP preparation into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

A dose of the VLP preparation administered can vary depending on various factors including, but not limited to, the weight, physical condition, and/or age of the subject, and/or the route of administration. Thus, the concentration of VLP in a given volume of the formulation can vary widely, and depends upon factors such as the species, age, weight and physical condition of the subject, and/or the method of administration. Accordingly, it is not practical to set forth generally the amount that constitutes an amount of the VLP preparation effective for all possible applications. Those of ordinary skill in the art, however, can readily determine the appropriate amount with due consideration of such factors.

In some embodiments, the method can include administering sufficient amount of a VLP preparation to provide a dose of, for example, from about 10⁹ VLPs/ml to about 10¹² VLPs/ml to the subject, although in some embodiments the methods may be performed by administering the VLP preparation in a dose outside this range. In some of these embodiments, the method includes administering sufficient VLP preparation to provide a dose of from about 4×10¹⁰ VLPs/ml to about 3×10¹¹ VLPs/m1to the subject, for example, a dose of from about 5×10¹⁰ VLPs/ml to about 1.2×10¹¹ VLPs/ml.

In some embodiments, the VLP preparation may be administered, for example, from a single dose to multiple doses per week, although in some embodiments the method can be performed by administering VLP preparation at a frequency outside this range. When multiple doses are used within a certain period, the amount of each dose may be the same or different. For example, a dose of 1 mg per day may be administered as a single dose of 1 mg, two 0.5 mg doses, or as a first dose of 0.75 mg followed by a second dose of 0.25 mg. Also, when multiple doses are used within a certain period, the interval between doses may be the same or be different.

In certain embodiments, the VLP preparation may be administered as a single one-off dose. In other embodiments, the VLP preparation may be administered from about once per week to about 15 times per day, such as, for example, from about once per day to about eight times per day. In certain embodiments, the VLP preparation may be administered three times per day.

In some embodiments, the VLP preparation may be administered after a sustained period of nutrient deprivation. As used herein, a “sustained period of nutrient deprivation” refers to a time longer than the usual time between meals. As used herein, “nutrient deprivation” refers to any reduced access to calories or nutrients. Thus, in some embodiments, a sustained period of nutrient deprivation can include fasting for a minimum period of at least eight hours such as, for example, at least 12 hours, and least 18 hours, at least 24 hours, at least 30 hours, or at least 36 hours. A sustained period of nutrient deprivation can include fasting for a maximum period of no more than 72 hours such as, for example, no more than 60 hours, no more the 54 hours, no more than 48 hours, no more than 42 hours, no more than 36 hours, no more than 30 hours, no more than 24 hours, or no more than 18 hours. In some cases, a sustained period of nutrient deprivation can include fasting for a period within a range having endpoints defined by any minimum fasting period set forth above and any maximum fasting period set forth above that is greater than the minimum fasting period. In certain embodiments, the sustained period of nutrient deprivation can include fasting for 12 hours to 36 hours. in one specific exemplary embodiment, the sustained period of nutrient deprivation can include fasting for 24 hours.

Nutrient deprivation reduces available food sources for resident gut bacteria, thus inhibiting the growth of gut bacterial populations. When used in conjunction with VLP treatment, nutrient deprivation can weaken resident gut bacteria thereby making administration of the VLP treatment more effective.

In some embodiments, the VLP preparation may be administered, for example, for a period ranging from a single dose to the remaining lifespan of the subject. In certain embodiments, the VLP preparation may be administered as a single one-off dose. In other embodiments, the VLP preparation may be administered for a period of one day to a period of about one year, such as, for example, for a period of from about three days to about seven days. Treatment with VLP preparation also may occur periodically as a retreatment to reduce the likelihood and/or severity of a recurrence of dysbiosis.

In some embodiments, VLPs are also known to encode for important genes (e.g., for metabolism). Therefore, transference of the VLP population can replace the recipient's VLP community and alter the metagenome, the transcriptome, and/or the metabolome of the recipient microbiome, replicating that of the environment from which the VLPs originated.

In some embodiments, gut dysbiosis-targeting VLPs may be collected, mixed, propagated, separated, enriched, purified, isolated and/or extracted from a variety of environmental samples that represent mixed microbial communities.

In some embodiments, artificial VLP communities may be constructed from any combination of phages, eukaryotic viruses, fungal viruses, archaeal viruses, and gene transfer agents.

While described above in the context of an exemplary embodiment, consistent with the experimental model, in which the VLPs were harvested from the gastrointestinal tract of mice, the compositions and methods described herein can involve VLP preparations obtained from any suitable microbiome of any suitable host or environment. Exemplary alternative hosts from which the VLP preparation can be prepared include, but are not limited to, for example, a human, a non-human primate, a dog, a cat, another mammal, a bird, a fish, an amphibian, a reptile, or a plant—e.g., a plant used in agriculture, botany, horticulture, aquaculture, hydroponics, or aquaponics. Exemplary environments from which the VLP preparation can be prepared include, for example, feces, blood, urine, sputum, phlegm, mucus, pus, sweat, lymph, semen, saliva, cerebrospinal fluid, tears, amniotic fluid, blood plasma, breast milk, synovial fluid, serum, vaginal lubrication, bile, aqueous humor, gastric acid, pre-ejaculate, earwax, transudate, vitreous body, chyme, chyle, sebum, rheum, colostrum, hemoglobin, smegma, salt water, soil, fresh water, sewage, activated sludge, hospital effluent, wastewater, treated wastewater, swamp water, marsh water, brackish water, lake water, pond water, stream water, river water, or a deep-sea vent.

In some embodiments, the method can further include administering to the subject a second composition for treating dysbiosis before, in conjunction with, or after VLP transplantation. The additional composition for treating dysbiosis can include, but is not limited to, a prebiotic, a probiotic, an antibiotic, a pharmaceutical or non-pharmaceutical pharmacological agent, a nutraceutical, a nutritional supplement, a biofilm modifier or emulsifier, an autophagy regulator, a phage-encoded protein, fecal or other microbiota transplantation, a dietary treatment, phage therapy, immunoglobulin therapy, interferon-gamma therapy, growth factor therapy, CRISPR-Cas9, or stem cell transplantation.

In some embodiments, the method can further include administering to the donor a composition for pre-treatment prior to collecting VLPs for transplantation. The additional composition for treating dysbiosis can include, but is not limited to, a prebiotic, a probiotic, an antibiotic, a pharmaceutical or non-pharmaceutical pharmacological agent, a nutraceutical, a nutritional supplement, a biofilm modifier or emulsifier, an autophagy regulator, a phage-encoded protein, fecal or other microbiota transplantation, a dietary treatment, phage therapy, immunoglobulin therapy, interferon-gamma therapy, growth factor therapy, CRISPR-Cas9, or stem cell transplantation.

Exemplary suitable prebiotics, probiotics, antibiotics, pharmaceutical agents, non-pharmaceutical pharmacological agents, nutraceuticals, nutritional supplements, biofilm modifiers and emulsifiers, autophagy regulators, phage-encoded proteins, microbiota transplants, and dietary treatments are listed, in detail, above.

In some embodiments, the method can include retreatment of the host and its associated microbiome to reduce the likelihood and/or severity of a recurrence or relapse of dysbiosis. This may involve regular treatments with VLPs with or without additional pharmaceutical compositions for treating dysbiosis. Regular treatments are exemplified by, but not limited to, treatments given at a periodic rate of biweekly, weekly, bimonthly, monthly, biannually, or annually.

Fecal microbiota transplants (FMTs) or the delivery of microbes or microbiota, in whole or in part, with desirable characteristics into the gastrointestinal tract of a recipient is another strategy for altering the gut microbial community by delivering the fecal gut microbiota collected from one or more donors into the gastrointestinal tract of a recipient. The efficacy of FMT—i.e., whether or not the transplanted microbiota takes up residence in the recipient—depends at least in part on the status of the gut microbial community of the recipient. Specifically, FMT can be highly effective when used in patients with diarrhea or colitis due to C. difficile whose native microbiota has been perturbed by antibiotics resulting in dysbiosis. In contrast, a gut microbiota that has not been perturbed would resist “take over” by “foreign” microbiota. However, individuals with an unperturbed microbiota could still benefit from FMT, particularly when the FMT delivers beneficial gut bacteria with desirable characteristics.

Since VLPs can reduce intestinal bacterial density by eliminating resident gut bacteria from the gastrointestinal mucosa, clinical treatment with VLPs also can improve the effectiveness of an FMT. In a clinical setting, FMTs are used as a treatment of antibiotic-induced Clostridium difficile infection; recipients with C. difficile dysbiosis respond to FMT therapy. Treatment of dysbiosis using FMT involves transferring fecal bacteria from a healthy donor into a recipient with dysbiosis. As a result, the donor's fecal bacteria colonize the intestine of the recipient, thereby resolving dysbiosis in the recipient. FMTs are successful approximately 85% of the time. Treatment with VLPs prior to, in conjunction with, or after the FMT will help eliminate the resident gut bacteria, which can improve the ability of the donor's bacteria to colonize the recipient's gastrointestinal tract. In addition, treatment with VLPs prior to, in conjunction with, or after the FMT can lead to increased diversity of the microbial community that is able to establish after FMT treatment by reducing the density of any particular bacterial strain or species that might otherwise outcompete others.

Thus, the VLP preparations described herein can improve the efficacy of fecal microbiota transplants in the setting of an unperturbed and intact gut microbiome, even when the individual is considered “healthy.” Administering a VLP transplant prior to FMT in recipients with an unperturbed, intact microbiome prepares or primes the recipient gut by disrupting, changing, and/or eliminating resident gut microbiota, enhancing the ability of the fecal microbial transplant to colonize the gut of the recipient.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Isolation of VLPs

Samples representing mixed microbial communities were collected from the feces of a C57L/6 mouse (Charles River, Wilmington, Mass.) as a source for VLPs. Samples were subjected to centrifugation, filtration, and concentration to enrich the VLP population. Initially, samples contained a diverse community of eukaryotes, prokaryotes, cellular debris, VLPs, as well as particulate matter. Centrifugation of the sample at 5000×g for 10 minutes pelleted most larger cells and particles. The VLP-containing supernatant was subsequently filtered through a 450 nm filter to remove any remaining bacteria. To concentrate the VLPs in the filtrate, a centrifugal filter with a 3 nm pore size was used, which is small enough to catch all known VLPs. After cleaning with several wash steps, the VLPs on the filter were resuspended by agitating the filter in a phosphate buffered saline (PBS) solution. The presence of VLPs, and the absence of other components in the “enriched VLPs”, was confirmed using fluorescent microscopy as previously described (Ortmann, AC and Suttle, Calif., 2009, Method Mol Biol 87-95).

### Electron Microscopy

VLPs in solution were also visualized to confirm their presence using electron microscopy. Transmission electron microscopy images were captured in the HSC-Electron Microscopy Facility, supported by the University of New Mexico Health Sciences Center. Briefly, 5-10 μL of the extracted fecal VLP fraction was incubated on glow-discharged, carbon-coated grids for five minutes, grids were then washed three times in ultrapure H₂O, excess liquid was wicked away, and the samples were stained for 1-2 minutes with 1% uranyl acetate. The excess stain was wicked away and grids were left to air dry. Samples were examined in a Hitachi HT7700 TEM operating at 80 kV. images were captured with an AMT XR-81 CCD camera. Exemplary results are shown in FIG. 6.

### Mouse Model for Dysbiosis

For the mouse model of gut dysbiosis, C57BL/6 mice (n=18) were fed a high fat diet (HFD) (70% of calories from lard). The HFD group (n=12) was given a 30-day treatment of HFD while the Control group (n=6) remained on a standard mouse diet (SD). After completing the 30-day HFD, the HFD group was switched back to a SD. After a 24-hour period, the HFD group was randomized into two groups for oral administration of either enriched VLPs (HFD-VLP; n=6) or a phosphate-buffered saline (PBS) control (HFD-PBS; n=6) via gavage; SD Control animals fed a standard diet received either enriched VLPs (SD-VLP; n=6) or PBS (Control; SD-PBS; n=6). PBS treatment served as the Control. Treatments were given once daily for three days. Mice were sacrificed 24 hours after the last treatment and tissue samples were collected from the distal most ⅓ or ileum of the small intestine.

To determine the ratio of bacteria in microbiome belonging to the Firmicutes and Bacteroidetes phyla, DNA extracted from the ileum of the small intestine was analyzed using separate primer pairs targeting Firmicutes-specific genes and Bacteroidetes-specific genes. Data were standardized against the 18s rRNA gene. Results are reported in FIG. 2 as the ratio of Firmicutes to Bacteroidetes, calculated using delta Ct values (mean±SE). The difference between means for each group was assessed with one-way ANOVA and the Uncorrected Fisher's LSD. Statistical analysis was performed with PRISM software (GraphPad Software, San Diego, Calif.).

To quantitate the density of the bacterial population, DNA was extracted from 25 mg of tissue using the DNeasy Blood and Tissue Kit (Qiagen) and then enumerated by quantitative polymerase chain reaction (qPCR) with a primer pair targeting the universal bacterial 16 s rRNA gene. As the 16 s rRNA gene is specific to bacteria and absent from the eukaryotic cells of the mouse host (identifiable by a different 18 s rRNA gene), this approach allows the specific enumeration of the bacterial members of the gut microbiome attached to the mucosa of the small intestinal tissue. 16 s rRNA gene count was then standardized against 18 s rRNA gene count to account for differences in the quantity of tissue that provided the extracted DNA. The delta delta Ct analysis was performed using the universal eukaryotic 18 s rRNA gene for this standardization. Results are reported in FIG. 3 as fold-change 16 s rRNA gene copy number (mean±SE) relative to the SD-fed Control group (at 1.0). The difference between means for each group was assessed with one-way ANOVA and the Uncorrected Fisher's LSD. Statistical analysis was performed with PRISM software (GraphPad Software, San Diego, Calif.).

To quantitate expression of the Muc2 protein in the ileal tissue, RNA was extracted using the RNeasy Mini Kit (Qiagen, Hilden, Germany) and converted to cDNA using the SuperScript III First-Strand Synthesis System (Invitrogen, Thermo Fisher Scientific, Carlsbad, Calif.). Muc2 expression is quantified using Muc2-specific primers and standardized against the 18 s rRNA gene. Results are reported in FIG. 5 as Muc2 gene copy number (mean±SE) relative to the SD-fed Control group (at 1.0). The difference between means for each group was assessed with one-way ANOVA and the Uncorrected Fisher's LSD. Statistical analysis was performed with PRISM software (GraphPad Software, San Diego, Calif.).

To characterize the composition of the gut bacterial community, copies of the 16 s rRNA gene in the extracted DNA were sequenced using the MiSeq platform (Illumina, Inc., San Diego, Calif.). Raw sequencing data was analyzed to identify differences in bacterial community composition between groups SD-PBS (n=4), SD-VLP (n=4), and HFD-VLP (n=4). This is done by comparing sequencing data against known bacterial 16 s rRNA sequences in the Greengenes database (Second Genome, Inc., South San Francisco, Calif.). This approach classifies each 16 s sequence in the extracted DNA sample as a known bacterial species. Using QIIME2 software (Boylen et al., 2018, Peer J Preprints 6:e27295v2), the community of bacterial species identified in each sample is compared using principal coordinates analysis. Briefly, this method summarizes the metadata from an entire bacterial community into two principal coordinates, which can then be plotted on an XY-coordinate plane (FIG. 4). Similarities or dissimilarities between groups can then be identified based on clustering of individuals within a group. Statistical differences between groups were assessed using permutational MANCOVA analysis.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A pharmaceutical composition comprising:
a preparation of heterogeneous virus-like particles (VLPs) obtained from the gastrointestinal tract of a subject; and
a pharmaceutically-acceptable carrier.

2. The pharmaceutical composition of claim 1, further comprising an adjuvant.

3. The pharmaceutical composition of claim 1, wherein the VLPs comprise bacteriophages, eukaryotic viruses, fungal viruses, archaeal viruses, gene transfer agents or a combination thereof.

4. A method of treating dysbiosis in a subject having, or at risk of having dysbiosis, the method comprising administering to the subject a virus-like particle (VLP) preparation in an amount effective to ameliorate at least one symptom or clinical sign of dysbiosis.

5. (canceled)

6. The method of claim 4, wherein the method further comprises administering to the subject a second pharmaceutical composition for treating dysbiosis.

7. The method of claim 6, wherein the second pharmaceutical composition for treating dysbiosis comprises an antibiotic, a prebiotic, a probiotic, a synbiotic, a microbiota transplant, a pharmaceutical agent, a non-pharmaceutical pharmacological agent, a nutraceutical, a nutritional supplement, a biofilm modifier, a biofilm emulsifier, an autophagy regulator, a phage-encoded protein, a dietary treatment, phage therapy, immunoglobulin therapy, interferon-gamma therapy, growth factor therapy, CRISPR-Cas9, or a stem cell transplant.

8. The method of claim 7, wherein the prebiotic comprises an fructo-oligosaccharide, a disaccharide, a monosaccharide, a polyol, pectin, a galacto-oligosaccharide, inulin, a short chain carbohydrate, a sugar alcohol, or oligofructose.

9. The method of claim 7, wherein the probiotic comprises a Pediococcus spp., a Streptococcus spp., a Lactococcus spp., a Lactobacillus spp., an Oenococcus spp., a Bifidobacterium spp., a Saccharomyces spp., an Enterococcus spp., an Escherichia spp., a Badllus spp., or a Leuconostoc spp.

10. The method of claim 7, wherein the synbiotic agent comprises a combination of a prebiotic and a probiotic.

11. (canceled)

12. The method of claim 4, wherein the dysbiosis is localized to epithelial tissue of the epidermis, a mucosal surface, at least a portion of the skin, at least a portion of the scalp, at least a portion of the oral cavity, at least a portion of a tooth, at least a portion of the gums, at least a portion of the gastrointestinal tract, at least a portion of the nasal cavity, at least a portion of the respiratory tract, at least a portion of the reproductive system, at least a portion of the circulatory system, at least a portion of the immune system, at least a portion of the genito-urinary tract, or a body cavity.

13-15. (canceled)

16. The method of claim 4, wherein the VLP preparation is prepared from an environmental sample.

17. The method of claim 16, wherein the environmental sample is obtained from salt water, soil, fresh water, sewage, activated sludge, hospital effluent, wastewater, treated wastewater, swamp water, marsh water, brackish water, lake water, pond water, stream water, river water, or a deep-sea vent.

18. The method of claim 16, wherein the VLPs in the environmental sample are enriched using an enrichment step.

19. The method of claim 4, further comprising a sustained period of nutrient deprivation of epithelial bacterial population prior to administering the VLP preparation to the subject.

20. (canceled)

21. The method of claim 4, wherein the VLP preparation is administered orally, administered by eye drops, administered during tooth brushing, administered by gargling, administered through inhalation, administered through a skin patch, applied or rubbed into the skin, applied into a wound, applied or rubbed onto a mucosal surface, enterally, rectally, administered through a delivery tube, administered through a medical device, nasally, or through a genito-urinary route.

22-28. (canceled)

29. The method of claim 4, wherein the VLP preparation is administered parenterally.

30. (canceled)

31. The method of claim 4, wherein the dysbiosis is localized to a plant-associated microbiome.

32. (canceled)

33. The method of claim 4, wherein the dysbiosis is associated with foodborne pathogens in a setting of food production or food processing.

34. (canceled)

35. (canceled)

36. A method of preparing or priming the gut environment of a healthy subject for administering a source of bacteria, the method comprising administering to the subject a virus-like particle (VLP) preparation in an amount effective to improve the receptivity of subject's gut to source of bacteria.

37. The method of claim 36, wherein the source of bacteria comprises a probiotic or a fecal microbiota transplant.

38. The method of claim 36, wherein the VLP composition is administered by mouth, delivered directly to the gut, or encapsulated as a microbial transplant.

39. The method of claim 36, further comprising a sustained period of nutrient deprivation of the subject's gut bacterial population prior to administering the VLP preparation to the subject.
